(19) United States
(12) Patent Application Publication
(10) Pub. No.: US 2013/0018268 A1
(43) Pub. Date: Jan. 17, 2013

(54) Scanning Speed Detection for Freehand High Frequency Ultrasound Transducers

(71) Applicants: Laust G. Pedersen, Santa Barbara, CA (US); Bobby Purkait, Santa Barbara, CA (US)

(72) Inventors: Laust G. Pedersen, Santa Barbara, CA (US); Bobby Purkait, Santa Barbara, CA (US)

(73) Assignee: NIVASONIX, LLC, Carpinteria, CA (US)

(21) Appl. No.: 13/546,871

(22) Filed: Jul. 11, 2012

Related U.S. Application Data
(60) Provisional application No. 61/506,771, filed on Jul. 12, 2011.

(51) Int. Cl. A61B 8/06

(52) U.S. Cl. 600/455

(57) ABSTRACT

Scanning speed detection for freehand high frequency ultrasound transducers using a multiple element moving array transducer using a pair of elements as a Doppler scanning speed detector. Various aspects of the invention are disclosed.

### CROSS-REFERENCE TO RELATED APPLICATIONS

This application claims the benefit of U.S. Provisional Patent Application No. 61/506,771 filed Jul. 12, 2011.

### BACKGROUND OF THE INVENTION

1. Field of the Invention

The present invention relates to the field of freehand high frequency ultrasound transducers.

2. Prior Art

A speed detection system for high frequency ultrasound transducers, open or closed loop, can take many forms, especially if the user is considered being part of the loop. Implementation with the user being in the loop is taught here and in U.S. Patent Application Publication No. 2010/0256489.

A quantified feedback based on one or more sensors can be used actively (closed loop), so the system for example automatically adjusts to a desired tissue temperature and/or emulsification rate for any scanning speed.

Three scanning speed sensing technologies, which have potential, are:

1. Doppler ultrasound sampled on tissue (as opposed to blood). See U.S. Pat. No. 5,127,409 entitled “Ultrasound Doppler position sensing”.

2. 3-axis accelerometers, position sensors or a GPS location system will be able to determine not only speed, but also location and can therefore be part of a scanning plan. See U.S. Pat. No. 6,290,649 entitled “Ultrasound position sensing probe”.

3. Optical location sensor technology similar to an optical PC mouse embedded in the ultrasound transducer. See

U.S. Pat. No. 7,532,201 entitled “Position Tracking Device”.

These sensor(s) can possibly also be used as skin contact detector(s).

### BRIEF DESCRIPTION OF THE DRAWINGS

FIG. 1 shows the resulting Doppler spectral responses from 4 angles of flow incidences to the transducer beams.

FIG. 2 shows a pre-curved multi element (10-element) 5-channel moving array transducer with only the outer elements active.

FIG. 3 is the expected Doppler spectrum from the moving transducer in FIG. 2.

FIG. 4 is a block diagram of Doppler speed sensing with user in feedback loop.

FIG. 5 is a block diagram of Doppler speed sensing with automated feedback loop.

### DETAILED DESCRIPTION OF THE PREFERRED EMBODIMENTS

The preferred embodiment of the present invention detects scanning speed utilizing Doppler Ultrasound. The Doppler equation is Fd=2*f*V* cos θ/c:

where Fd is the Doppler frequency,

f is the transmitted ultrasound frequency,

V is the scanning speed,

θ is the angle between the scanning velocity and beam direction and

c is the speed of sound in tissue, 1500 m/sec.

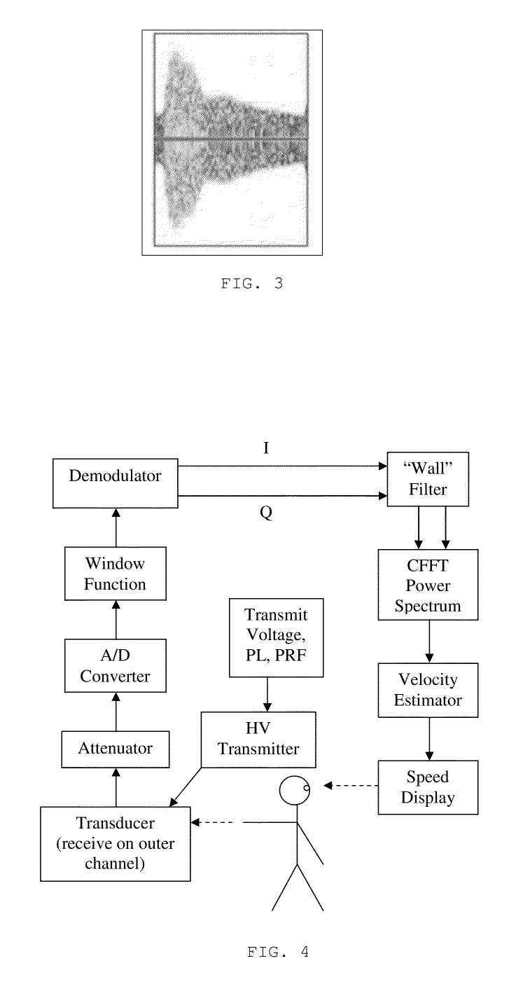

If the F-number (focal length divided by the aperture size) is 1.0 for the HIFU transducer, then the angle a in FIG. 2 is A tan (0.5)=26.6 degrees.

The freehand scanning speed range may be between 0.2 and 20 cm/sec, typically 1 to 5 cm/sec. Using the Doppler equation and an F1 transducer aperture, a 1 MHz transducer will typically show peak spectra of 5.8 to 29 Hz and a 7 MHz transducer will show peak spectra of 41 to 203 Hz for the typical scanning speeds.

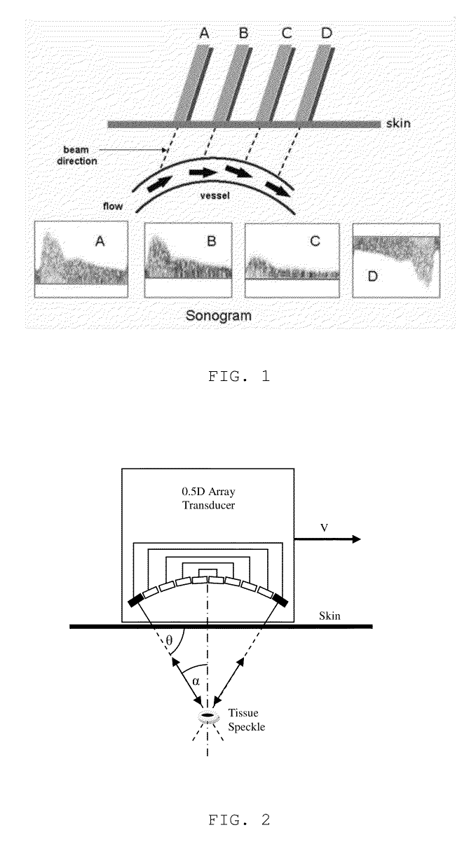

In FIG. 1, the Doppler spectrum A comes from transducer A, whose beam is intersected by a blood flow only toward the transducer, hence the positive Doppler spectrum. The B and C spectra are from transducers with beam-flow intersection angles approaching 90 degrees, hence lower spectra. The D spectrum is negative since the blood flows away from transducer D where its beam intersects the blood flow.

FIG. 2 shows a cross-section of the preferred 0.5D brush-type HIFU transducer (see U.S. Pat. No. 7,955,281 for a 3-channel design example, the disclosure of which is hereby incorporated by reference), with the outer (spaced apart) elements (connected in parallel and) used as a pulsed Doppler transducer. In the preferred embodiment these Doppler elements may be the HIFU elements as well and the HIFU burst can be used as the transmitting Doppler burst. For better Doppler angle the Doppler sample volume needs to be close to the transducer, which means that the transducer may simultaneously transmit and receive. This will result in a RF signal, which is a mix between continuous wave (CW) and pulsed wave (PW) Doppler.

The transmitted bursts are reflected by the speckle (constellation of scatters in tissue, which sums up coherently to a large reflection) which is always present in an ultrasound image as the transducer is moved to the right at a speed of V.

The typical concern in CW Doppler processing is the large amplitude difference between the transmit and receive signals. However, since the returned signal from tissue is much larger than from the low reflecting blood (by about 20 dB), the transmit/receive ratio will be much smaller and therefore processing for this type of Doppler scanning speed detection can be simplified.

It should be realized that many other schemes can be implemented with success, such as 1) continuously transmitting a Doppler signal low enough and/or phased not to substantially adversely affect the HIFU beamforming, 2) timed low level Doppler pulses to occur in between and preferably just before the HIFU pulses, where the system is most “quiet” and 3) add dedicated Doppler element(s) to the transducer not used for HIFU beamforming.

FIG. 3 shows the Doppler spectrum (spectrogram) resulting from the moving transducer shown in FIG. 2. In this case the outer element pair (outer channel) is being used as Doppler transducers and the speed estimator can be based on the positive and negative peaks of the Doppler spectrum. The left outer transducer element sees tissue moving toward it, hence a positive Doppler spectrum and the right outer transducer element sees tissue moving away from it, hence a negative Doppler spectrum. The net effect of the simultaneous positive and negative spectra is displayed in FIG. 3. With the help of the Doppler equation the scanning speed as a first approximation can be found from the peak to peak value of the Doppler spectrum. In a more refined analysis the beam profiles of the elements need to be accounted for as well. As an alternative, a Doppler spectrum shape to scanning speed can be empirically established, once the transducer and Doppler system is reduced to practice.

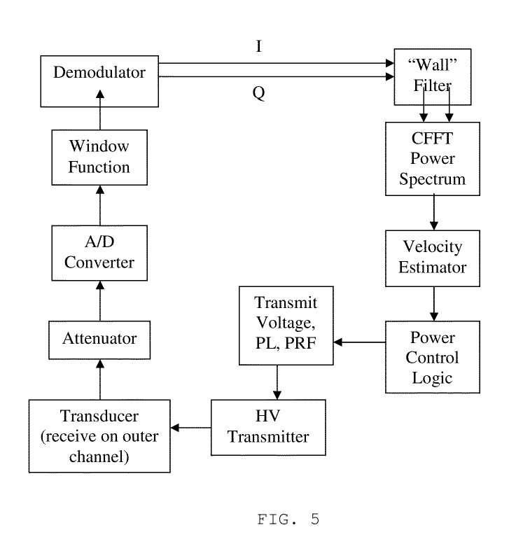

FIG. 4 is a block diagram of the Doppler based speed sensor with the user in the feedback loop tasked with moving the transducer at a predetermined speed. A high voltage (HV) transmitter energizes an ultrasound transducer array to transmit ultrasound bursts, which are fired repeatedly at a pulse repetition frequency (PRF) so that ultrasound waves are backscattered from a predetermined volume of tissue from a subject under study. The transmitter unit includes a control for adjusting the delivered power by controlling any combination of the PRF, pulse (burst) length (PL) and transmit voltage (Vt). The Power delivered is proportional to the PL and the PRF and proportional to the Vt squared.

The return RF signals are detected by the transducer's outer channel, two outer elements electrically connected in parallel. The signal is first attenuated, then digitized by an A/D converter. A window function (rectangle, Hamming, Hanning, Gaussian, Blackman-Harris, or other) is then applied to each return, then demodulated by a demodulator into its in-phase and quadrature (I/Q) components.

The I/Q components are digitally (high pass) filtered, labeled “wall filter” since it traditionally filters the vessel wall motion out. However in this case, where the tissue is in motion relative to the transducer, the filter is adjusted to exclude frequencies below the lowest relevant frequency (slowest speed).

The filtered signals are transformed to the frequency domain by applying Complex Fast Fourier Transforms (CFFTs) over a moving time window of typically 64 to 128 samples. The result is a frequency spectrum as a function of time similar to the spectrogram shown in FIG. 3.

From this a speed estimator, which could be a simple as a scaled peak to peak detector is applied to the spectrogram. The transducer speed information is then displayed on the system, which is observed by the user, who in turn adjusts the transducer scanning speed to a predetermined speed.

FIG. 5 is identical to FIG. 4 except that the feedback loop here is automatic.

The user can use any scanning speed within a certain range, typically 1 to 5 cm/sec, and the system will compensate to give the desired tissue heating by increasing the delivered power if the scanning speed is fast and decreasing the power if the scanning speed is slow. The measured scanning speed is read by the “power control logic” module, which determines what the power setting should be, by adjusting any of the three power controlling parameters (PRF, PL and Vt).

If the therapeutic effect is based on cavitation, there also needs to be a cavitation detector controlling the transmit voltage Vt, so in this case the PRF and/or PL need to compensate for both transducer speed and instantaneous transmit voltage supplied in order to maintain constant energy delivery to the tissue of interest.

### Automatic Feedback Control

In an automatic closed loop system for a HIFU transducer used for tissue heating, the peak temperature generally depends on the energy dose delivered to the tissue volume of interest. If the transducer is static the tissue volume will heat up until thermal equilibrium is obtained, power supplied equals power drained through tissue conduction and blood perfusion. If the transducer is moving, the energy is only delivered while the beam passes the tissue volume of interest. So in order to obtain the same temperature as in the static case the power delivery needs to increase with increasing scanning speed.

The fact that the static case requires the lowest power could make a closed loop scanning speed detector (SSD) an adequate skin contact detector a well, since the detector does not sense motion if the transducer is lifted from the skin and therefore the power delivered may be low enough to not have any side effects, such as transducer overheating.

In case the HIFU transducer is used for therapy (for example fat emulsification) emulsification based on cavitation with a cavitation detector as well, the SSD can still be used to control the power delivery, but in this case it should control the duty cycle only to affect the power. The cavitation detection should only control the transmit voltage (proportional to the pressure in tissue and when squared proportional to the power delivered). In this case the SSD can maintain a desired tissue temperature in the region of interest for the therapy intended to give a consistent and uniform treatment outcome.

Thus the present invention has a number of aspects, which may be practiced alone or in various combinations or sub-combinations, as desired. While a preferred embodiment of the present invention has been disclosed and described herein for purposes of illustration and not for purposes of limitation, it will be understood by those skilled in the art that various changes in form and detail may be made therein without departing from the spirit and scope of the invention as defined by the full breadth of the following claims.

What is claimed is:

1. A method of scanning speed detection for freehand high frequency ultrasound transducers using a multiple element moving array transducer comprising:
using a pair of elements as a Doppler scanning speed detector.

2. The method of claim 1 wherein the pair of elements of the freehand high frequency ultrasound transducer are outer elements electrically connected in parallel.

3. The method of claim 1 wherein the Doppler scanning speed detector uses Doppler bursts.

4. The method of claim 3 wherein the pair of elements are also used as freehand high frequency ultrasound transducer elements.

5. The method of claim 4 wherein the Doppler bursts are between high frequency ultrasound transducer pulses.

6. The method of claim 5 further determining the scanning speed using an empirically determined scanning speed to Doppler spectrum shape.

7. The method of claim 5 wherein the delivered power to the freehand high frequency ultrasound transducer is controlled by controlling any combination of the pulse repetition frequency, the pulse burst length and the transmit voltage.

8. The method of claim 2 wherein the return signals are digitized.

9. The method of claim 8 wherein a window function is applied to the digitized return signals, then demodulated into its inphase and quadrature components.

10. The method of claim 9 wherein the inphase and quadrature components are transformed to the frequency domain by applying Complex Fourier Transforms over a moving time window to obtain a frequency spectrum as a function of time.

11. The method of claim 10 wherein a speed estimator is applied to the frequency spectrum as a function of time to provide an estimate of the scanning speed.

12. The method of claim 11 wherein the power delivered to the ultrasound transducer is automatically adjusted for differences in scanning speed by controlling any combination of pulse repetition frequency, pulse burst length and ultra sound transducer transmit voltage.

13. The method of claim 11 further comprising using a cavitation detector to detect cavitation to control the transmit voltage.

14. The method of claim 13 wherein transmit voltage is controlled by the cavitation detector, and the power delivered to the ultrasound transducer is automatically adjusted for differences in scanning speed by controlling any combination of pulse repetition frequency and pulse burst length.
